(19) United States
(12) Patent Application Publication
(10) Pub. No.: US 2011/0098219 A1
(43) Pub. Date: Apr. 28, 2011

(54) System for Curing Hepatic Cancer with Leptin

(75) Inventors: Shen-Nien Wang, Kaohsiung City (TW); Yao-Tsung Yeh, Kaohsiung City (TW); King-Teh Lee, Kaohsiung City (TW); Hsiang-Jen Yang, Chaozhou Township (TW); Sheau-Fang Yang, Kaohsiung City (TW)

(73) Assignee: KAOHSIUNG MEDICAL UNIVERSITY, Kaohsiung City (TW)

(21) Appl. No.: 12/689,718

(22) Filed: Jan. 19, 2010

(30) Foreign Application Priority Data
Oct. 22, 2009 (TW) 098135723

(51) Int. Cl. A61K 31/57; A61K 38/22; A61P 35/00; A61P 1/16

(52) U.S. Cl. 514/5.8; 514/178

(57) ABSTRACT

The present invention cures hepatic cancer by MPA with the help of leptin. A therapeutic drug of MPA combined with leptin is applied to a liver cancer patient. Or, MPA can be directly applied to a patient having a high leptin expression. Or, if a patient has a low leptin expression, MPA is applied to the patient after leptin expression is increased. Hence, expression of leptin can be taken as a predictive factor and a prognostic factor of treatment effect on curing the patient with MPA.

### FIELD OF THE INVENTION

The present invention relates to curing hepatic cancer; more particularly, relates to a hepatic cancer curing system, where medroxyprogesterone acetate (MPA) is combined with leptin for poisoning liver cancer cells effectively.

### DESCRIPTION OF THE RELATED ART(S)

Liver cancer has a serious influence to human life. Traditionally, through surgery, liver cancer is cured or survival rate of a liver cancer patient is improved. But, only 10˜15% of the patients can be handled with surgery, while the other patients are treated through radiotherapy, chemotherapy or immunotherapy for improving survival rate. However, all of their therapeutic effects are not so good, where radiotherapy and chemotherapy may cause harm to the patient with possible unwanted side-effects.

Recent search suggests that an adipokine generated by adipocyte, called leptin, is correlated to occurrence of liver cancer. It is confirmed that some cancers are correlated to leptin, including endometrial cancer and breast cancer. It is pointed out that leptin is interacted with specific receptors on membrane to modulate expression of some genes through transferring STAT3. And, some of the genes, like vascular endothelial growth factor (VEGF), are related to occurrence of cancer.

In recent clinical researches, hormone therapy is increasingly used for curing liver cancer patients, where liver cancer is found to be a sex-hormone dependant tumor—mostly related to androgen and obviously related to steroid hormone and changes of sex hormone receptor. A new therapy using a glucocorticoid antagonist is invented. For example, progesterone and RU486 can suppress expression of α-fetoprotein in human liver. It is possible to use progesterone for hormone therapy on liver cancer.

Modern researches show that MPA can effectively suppress growth of liver cancer cells. MPA is derived from 17-OH-progesterone, which has a structure similar to natural luteining hormone with differences on methyl group at position of α-6 and acetoxy group at position of 17. MPA has antiestrogenic activity and can suppress the levels of serum estrogen in postmenopausal breast cancer. MPA also suppresses release of luteining hormone (LH); stimulates growth of endometrium; and causes typical hormone changes in acinar cells of breast. Although mechanism of MPA is not very clear, interactions among hormone receptors is believed to have something to do with MPA. In other researches, it is found that MPA can be interacted with receptors of estrogen, luteining hormone and androgen, where the interaction with androgen receptor is especially important to the toxicity to cells. Recent studies also found that MPA could suppress the size of liver tumor and might improve survival rate of liver cancer patient. Related researches further confirm that MPA may affect development of liver cancer and thus improve patient's prognosis. Hence, MPA may aid in the survival rate of liver cancer patients for clinical treatment. However, MPA has multiple pharmacologic activities and further studies on their mechanisms are necessary.

Some clinical experiments prove that MPA has a suppressive effect on liver cancer patients at telophase. Related researches also prove its suppression on growth of hepatocellular carcinoma cell line (HepG2 cells). Yet, the prior researches did not find significant association of patient's survival rate with leptin expression in liver cancer tissue, and MPA treatment after surgery (P values of 0.383 and 0.171 is shown in FIGS. 19A and 19B, respectively). Moreover, it is proved that the micro vascular density in liver cancer tissue and the expression of Ki-67 have no significant relationship to patient's survival time.

As a conclusion, leptin is a hormone of single-chain protein and is generally considered to play an important role on maintaining body weight and adjusting body temperature; and, MPA is a composed steroid derived from luteining hormone. Researches until now prove that MPA has effect on causing apoptosis of hepatocellular carcinoma cell line in vivo and in vitro. That is, MPA can effectively suppress growth of hepatocellular carcinoma cell line. But, in related researches also found that MPA has no obvious effect on improving patient's survival rate. Although MPA is already a drug for cancer in market, there is still no exciting treatment effect found for liver cancer patients. MPA has not yet obtained statistical difference on survival rate of liver cancer patients. Besides, it is not found to apply MAP according to expression of a bio-mark in patient's serum or liver cancer tissue, like leptin. Hence, the prior arts do not fulfill all users' requests on actual use.

### SUMMARY OF THE INVENTION

The main purpose of the present invention is to enhance MPA's suppression on liver cancer cells by using leptin.

The second purpose of the present invention is to directly apply MPA to a liver cancer patient with enhanced pharmacologic effect while the patient has a high leptin expression.

The third purpose of the present invention is to apply MPA combined with leptin (MPA+leptin) to a liver cancer patient with enhanced pharmacologic effect while the patient has a high leptin expression.

The fourth purpose of the present invention is to, after raising expression of leptin with 5-hydroxy-trypton (5-HTP) in a liver cancer patient having a low leptin expression, apply MPA to the patient with enhanced pharmacologic effect.

To achieve the above purposes, the present invention is a system for curing hepatic cancer with leptin, comprising a therapeutic drug, a therapeutic procedure and a screening platform, where the therapeutic drug is a cytotoxic agent of MPA or MPA combined with leptin (MPA+leptin); leptin is obtained by adding leptin from outside of a patient or increasing physiological concentration of leptin inside the patient; an effective dose of the therapeutic drug is applied to the patient to contact with liver cancer cells to enhances and accelerates poisoning liver cancer cells by MPA with leptin through an interaction of a leptin receptor and a progesterone receptor; MPA+leptin has a pharmacologic specificity on epithelial cell line of normal liver and cell line of liver cancer; and the screening platform uses expression of leptin as a predictive factor and a prognostic factor of treatment effect on curing the patient with MPA. Accordingly, a novel system for curing hepatic cancer with leptin is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the view showing the prognostic survival time according to the preferred embodiment of the present invention;

FIG. 2 is the view showing the leptin's effect on the growth of liver cancer cells;

FIG. 3 is the view showing the MPA's effect on the growth of liver cancer cells;

FIG. 4 is the view showing the effect of MPA+leptin on the growth of liver cancer cells;

FIG. 5 is the view showing the statuses of the cells after applied with MPA+leptin;

FIG. 6A and FIG. 6B are the views showing the effect of MPA+leptin on the cell cycle;

FIG. 7 is the view showing the effect of MPA+leptin on the related proteins for apoptosis;

FIG. 8A and FIG. 8B are the views showing the interaction between the leptin receptor and the progesterone receptor through immunoprecipitation and immunofluorescent staining;

FIG. 9 is the view showing the reduced effect of MPA+leptin by knockdowning the leptin receptor expression using leptin receptor-specific siRNA;

FIG. 10A is the view showing the apoptosis on increasing the expression of leptin receptor using overexpression of ob-Rb plasmid;

FIG. 10B is the view showing the apoptosis on increasing the expression of progesterone receptor using overexpression of PR plasmid;

FIG. 11 is the view showing the expression of JAK/STAT path after applied with the drug;

FIG. 12 is the view showing the expression of MAPK path after applied with the drug;

FIG. 13A is the view showing the expressions of proteins of JAK/STAT path after applied with the drug for a short time;

FIG. 13B is the view showing the expressions of proteins of MAPK path after applied with the drug for a short time;

FIG. 14 is the view showing the reduced STAT3 activation by suppressing the ERK1/2 activation and the PIAS3 expression;

FIG. 15A is the view showing the cells overexpressing leptin with transfection with leptin plasmid;

FIG. 15B is the view showing the suppressed growth of the cells by using MPA;

FIG. 16A is the view showing the STAT3-related signal path of the cells after MPA is applied;

FIG. 16B is the view showing the MAPK-related signal path of the cells after MPA is applied;

FIG. 17A is the view showing the epithelial cell line of normal liver treated with MPA+leptin for 24 hours;

FIG. 17B is the view showing the epithelial cell line of normal liver treated with MPA+leptin for 48 hours;

FIG. 18A is the view showing the STAT3-related signal path after treating the cell line of normal liver with MPA+leptin;

FIG. 18B is the view showing the MAPK-related signal path after treating the cell line of normal liver with MPA+leptin;

FIG. 19A is the view of the relationship between the patient survival time and the leptin expression; and

FIG. 19B is the view of the relationship between the patient survival time and the postoperative MPA treatment.

### DESCRIPTION OF THE PREFERRED EMBODIMENT

The following description of the preferred embodiment is provided to understand the features and the structures of the present invention.

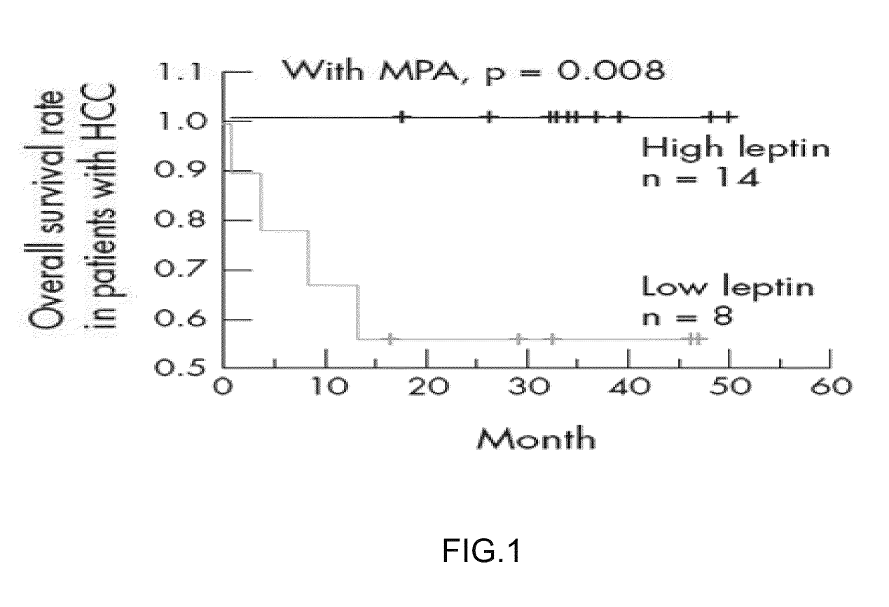

Please refer to FIG. 1, which is a view showing prognostic survival time according to a preferred embodiment of the present invention. As shown in the figure, the present invention is a system for curing hepatic cancer with leptin, which provides statistics of hepatocellular carcinoma cell line and liver cancer patients. The preferred embodiment comprises a therapeutic drug, a therapeutic procedure and a screening platform. Therein, the therapeutic drug is a cytotoxic agent of medroxyprogesterone acetate (MPA) or a cytotoxic agent of MPA combined with leptin (MPA+leptin), where leptin is obtained by being added from outside of a patient or increasing leptin's physiological concentration inside the patient. The therapeutic procedure applies an effective dose of the therapeutic drug to the patient to contact with liver cancer cells, where leptin enhances and accelerates poisoning liver cancer cells by MPA through an interaction of a leptin receptor and a progesterone receptor to obtain a pharmacologic specificity on epithelial cell line of normal liver and cell line of liver cancer. The screening platform uses expression of leptin as a predictive factor and a prognostic factor for treatment effect on curing the patient with MPA. Thus, the therapeutic drug reduces size and quantity of cell line of liver cancer to effectively improve survival rate of liver cancer patients while preventing bad side-effect of poisoning normal liver cells.

In ‘in vitro’ studies and clinical experiments, leptin and MPA are found to have obvious effect on suppressing liver cancer cells and improving patient's survival rate. Thus, the therapeutic drug is formed by combining MAP with leptin to cure liver cancer patients, where the physiological concentration of leptin in a normal human body is almost able to enhance the treatment effect of MPA. MPA is used for treatment in the present invention, where, for obtaining a better effect, leptin is directly combined with MPA or leptin is obtained by increasing the physiological concentration of leptin in the patient at first. Statistics obtained from clinical liver cancer patients show that, in the liver cancer patients treated with MPA after surgery, the patients having higher leptin expression have longer survival time than those having lower leptin expression, where P value is 0.008.

Thus, it shows that a patient having a higher leptin expression has a better survival rate than a patient having a lower leptin expression, which is obtained by treating the patients with a hormone therapy (i.e. MPA) after surgery. Hence, the leptin expression can further become a predictive factor for the treatment effect of MPA and a prognostic factor for survival time, where a higher leptin expression obtains a more obvious suppression on liver cancer cells by MPA. Consequently, the leptin expression can be taken as an index for effectiveness of the hormone therapy (i.e. MPA) after the surgery; and, the survival time of the liver cancer patients can be maintained or even prolonged.

As a conclusion, a patient having a higher leptin expression obtains a better treatment effects with MPA than a patient having a lower leptin expression; and the survival rate may be prolonged for five years. Besides, in vitro experiments for the present invention confirm that leptin can enhance MPA's suppression on liver cancer cells. Thus, the present invention not only provides a therapeutic drug and a therapeutic procedure with MAP+leptin; but also provides a screening platform with leptin to decide MPA treatment.

Concerning the mechanism of MPA+leptin applied on liver cancer cells, hepatocellular carcinoma cell line is taken to be treated with MPA+leptin for observing their survival rate; and, their cell cycles, apoptosis, working mechanisms and related signal paths are analyzed. In addition, pharmacologic effects of MPA+leptin on epithelial cell line of normal liver is examined to further obtain effect of hormone therapy on liver cancer by observing specificity of the drug.

### [State 1] Effect of Leptin on Growth of Liver Cancer Cell

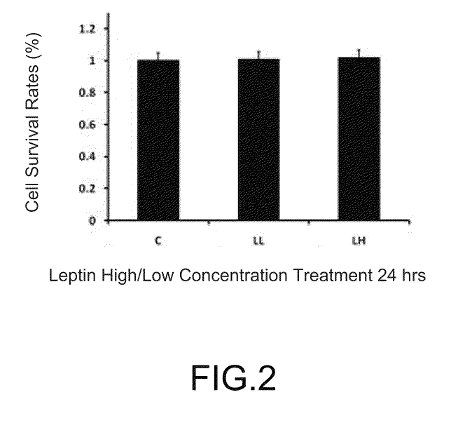

Please refer to FIG. 2, which is a view showing leptin's effect on growth of liver cancer cells. As shown in the figure, for acquiring effect of leptin on liver cancer cells, the preferred embodiment uses leptin having different doses, where the high-dose leptin has 100 nano-grams (ng) and the low-dose leptin has 10 ng. The 10 ng leptin is conformed to the physiological concentration of normal human body for observing effect of leptin on the cells. In the other hand, the 100 ng leptin is used to show whether a higher levels of leptin obtains a higher effect on the cells. As results show, after the cells are applied with high-dose leptin for 24 hours, leptin has no obvious effect on the cells.

### [State 2] Effect of MPA on Growth of Liver Cancer Cell

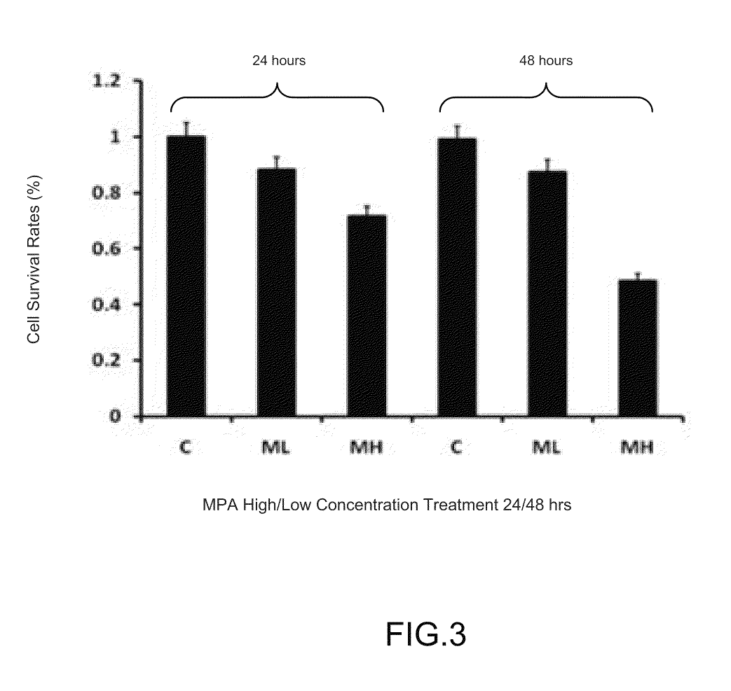

Please refer to FIG. 3, which is a view showing MPA's effect on growth of liver cancer cells. As shown in the figure, the preferred embodiment applies MPA having different doses to liver cancer cells. The high-dose MPA has a dose of 10⁻⁴ moles (M) and the low-dose MPA has a dose of 10⁻⁶ M. By analyzing experimental results through XTT assay, the high-dose MPA has obvious suppressive effect on the cells after being applied for 24 hours and the effect is enhanced after being applied for 48 hours. However, the low-dose MPA does not have obvious suppressive effect on the cells after being applied neither for 24 hours nor 48 hours. Thus, only a higher dose MPA obtains a better suppressive effect on liver cancer cells.

### [State 3] Effect of MPA+Leptin on Growth of Liver Cancer Cell

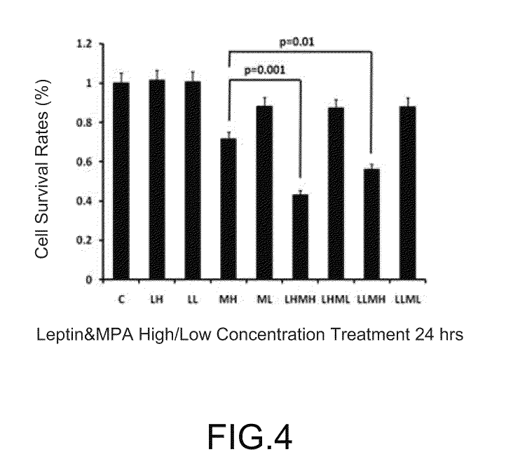

Please refer to FIG. 4, which is a view showing effect of MPA+leptin on growth of liver cancer cells. As shown in the figure, the preferred embodiment applies MPA combined with leptin to liver cancer cells, where MPA has different doses and leptin has different doses. As results show through an analysis of XTT assay, a better suppressive effect is obtained for liver cancer cells after being applied with the high-dose MPA combined with the high-dose leptin for 24 hours than that obtained by applying MPA only—P value is 0.001. The same is that a better suppressive effect is obtained for liver cancer cells after applying high-dose MPA combined with low-dose leptin than that obtained by applying MPA only—P value is 0.01. However, no obvious suppressive effect on liver cancer cells is obtained after applying low-dose MPA combined with low- or high-dose leptin. Hence, leptin is confirmed to have effect on enhancing suppression on liver cancer cell by MPA; in addition, the higher dose leptin, the more effective suppression.

### [State 4] Effect of MPA+Leptin on Apoptosis of Liver Cancer Cell

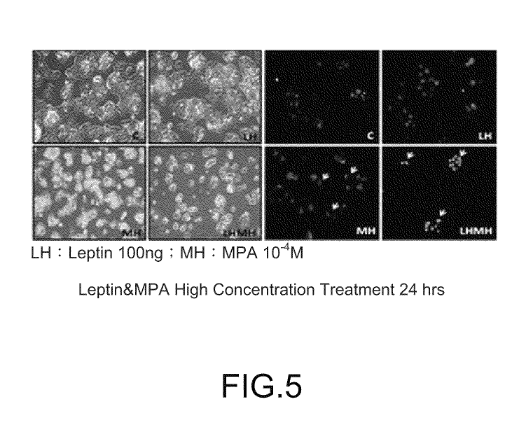

Please refer to FIG. 5, which is a view showing statuses of cells after applied with MPA+leptin. As shown in the figure, from the above State 3, leptin is confirmed to have effect on enhancing suppression on liver cancer cells. The preferred embodiment shows statuses of cells. After applied with MPA+leptin, apoptosis of the cells is more obvious than that obtained by applied with MPA only. The preferred embodiment further shows the statuses of cells through immunofluorescence. After applied with MPA+leptin for 24 hours, nuclei of the cells are dyed with DAPI to be observed with fluoresce microscope at 40×. The observation shows the same that atrophy of the nuclei are more obvious than that obtained by applying MPA only. Thus, it is confirmed that leptin is able to enhance and accelerate apoptosis of liver cancer cells by MPA.

### [State 5] Effect of MPA+Leptin on Cell Cycle

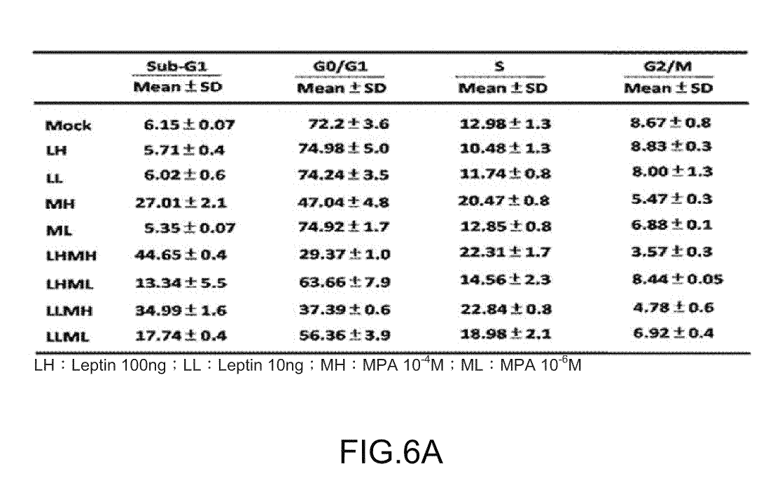

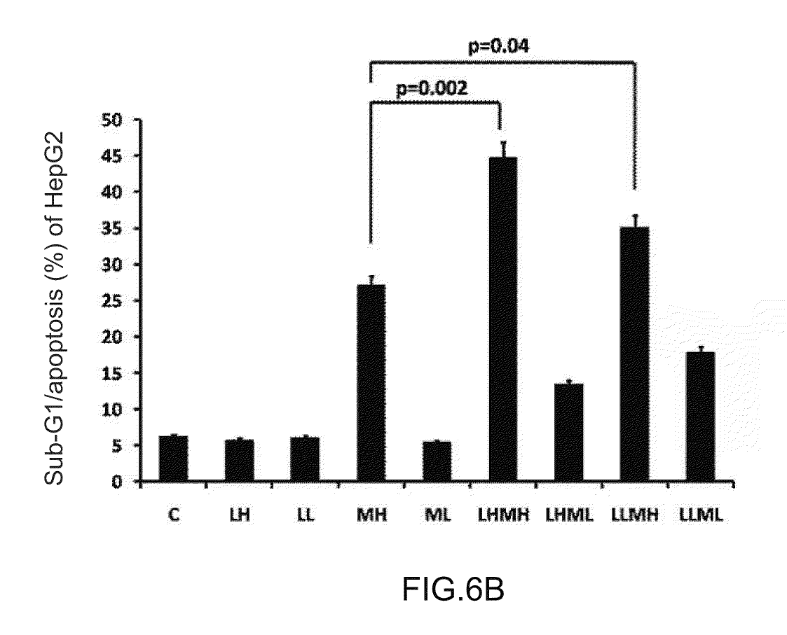

Please refer to FIG. 6A and FIG. 6B, which are views showing effect of MPA+leptin on cell cycle. As shown in the figures, for further recognizing role of MPA+leptin for liver cancer cells, the preferred embodiment applies leptin only, MPA only and MPA+leptin on liver cancer cells for 24 hours and a flow cytometry is used to show their effects on cell cycle. Furthermore, sub-G1/apoptosis parts in the cell cycle are analyzed through student's t-test. As results show, the cells obtained after applied with MPA has an increasing trend in sub-G1, which means dead cells are obviously increased; yet, has a decreased trend in G1 and G2/M and an increased trend again in S. For high-dose MPA combined with high-dose leptin, sub-G1 has a better increasing trend after 24 hours than that obtained by applying MPA only—P value is 0.002. For high-density MPA combined with low-density leptin, sub-G1 also has a better increasing trend than that obtained by applying MPA only—P value is 0.004; yet, has a decreased trend in G1 and G2/M and an increased trend in S. Hence, it is confirmed that MPA+leptin has better effect on apoptosis of liver cancer cells than MPA only; and MPA+leptin mainly enhances MPA's suppression in G1 and G2/M; or stops the cell cycle at S.

### [State 6] Analysis of Related Protein for Apoptosis

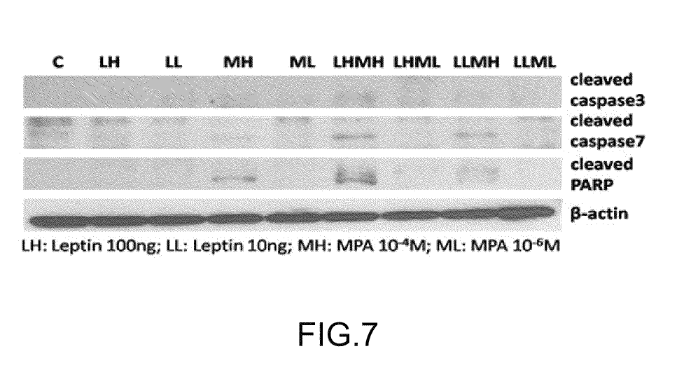

Please refer to FIG. 7, which is a view showing effect of MPA+leptin on related proteins for apoptosis. As shown in the figure, through analysis on liver cancer cells after applied with MPA+leptin for 24 hours, observation is done with a focus on changes of their related proteins for apoptosis. When different doses of MPA are combined with different doses of leptin for treatment for 24 hours, cellular proteins of anti-cleaved caspase 3, anti-cleaved caspase 7 and anti-cleaved Poly-ADP-ribose-polymerase (PARP) are processed with western blotting. Thus is observed that cleaved caspase 3/7 and cleaved PARP are obviously activated by MPA only; and, cleaved caspase 3/7 and cleaved PARP are more obviously activated by MPA+leptin than by MPA only.

### [State 7] Interaction Between Leptin Receptor and Progesterone Receptor

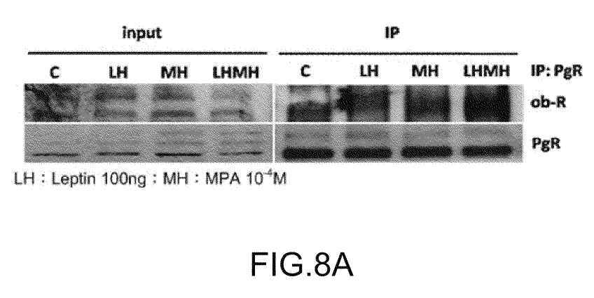

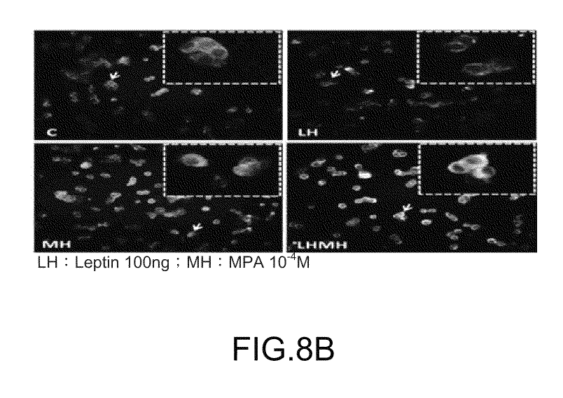

Please refer to FIG. 8A and FIG. 8B, which are views showing interaction between a leptin receptor and a progesterone receptor through immunoprecipitation and immunofluorescent staining. As shown in the figures, for acknowledging interaction between a leptin receptor and a progesterone receptor, cellular proteins are processed through immunoprecipitation and western blotting with anti-pgR after liver cancer cells are applied with leptin only, MPA only and MPA+leptin for 30 minutes separately. As results show, after applied with MPA only for 30 minutes, the leptin receptor and the progesterone receptor are interacted; and, after applied with MPA+leptin for 30 minutes, the interaction of leptin receptor and the progesterone receptor are more obviously increased as shown In FIG. 8A. The same results are shown through immunofluorescent staining. After the cells are processed for 10 minutes, the progesterone receptor is dyed with FITC; the leptin receptor is dyed with Texas-Red; and cell membrane is dyed with phalloidin, which are observed with fluoresce microscope under 40×. When only MPA is applied, some of the leptin receptor are merged with some of the progesterone receptor; and, when MPA+leptin is applied, the merged level between the leptin receptor and the progesterone receptor is more obvious as shown in FIG. 8B. Thus, it is confirmed that the leptin receptor and the progesterone receptor interact to work on the liver cancer cells.

### [State 8] Reduced Effect of MPA+Leptin by Suppressing Leptin Receptor Expression

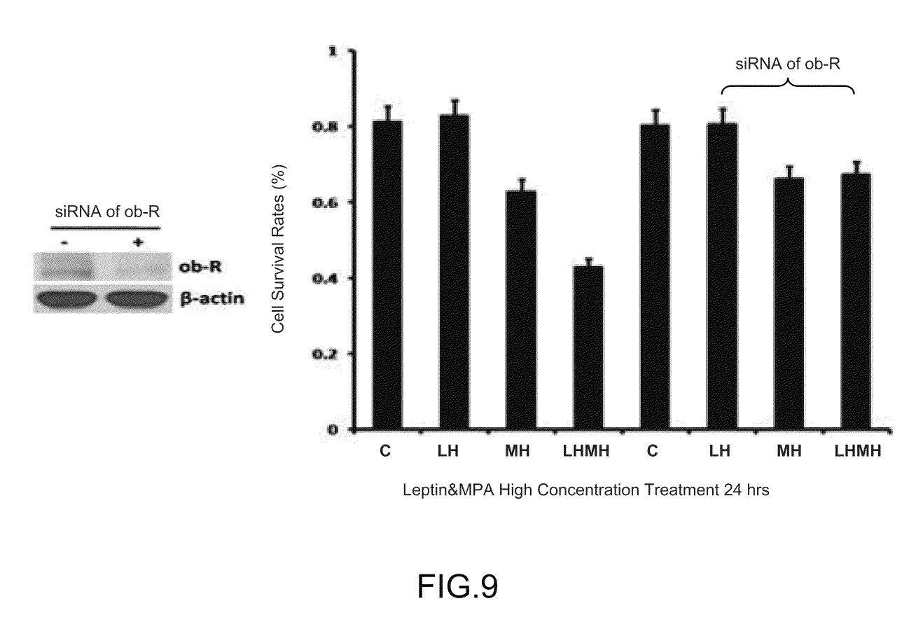

Please refer to FIG. 9, which is a view showing reduced effect of MPA+leptin by suppressing leptin receptor expression. As shown in the figure, a 20 nano-moles (nM) of leptin receptor, siRNA, is transferred to cells to be cultured a day and is cultured in a serum-free medium for the next day. Then, the cells are processed with MPA+leptin for 24 hours to be analyzed through XTT assay. As results show, when expression of the leptin receptor of siRNA is reduced, leptin does not enhance suppression of MPA on the cells. Thus, it is confirmed that leptin enhances MPA's suppression on the cells through the leptin receptor.

### [State 9] Enhanced Apoptosis of Cells by Increasing Expression of Leptin Receptor and Progesterone Receptor

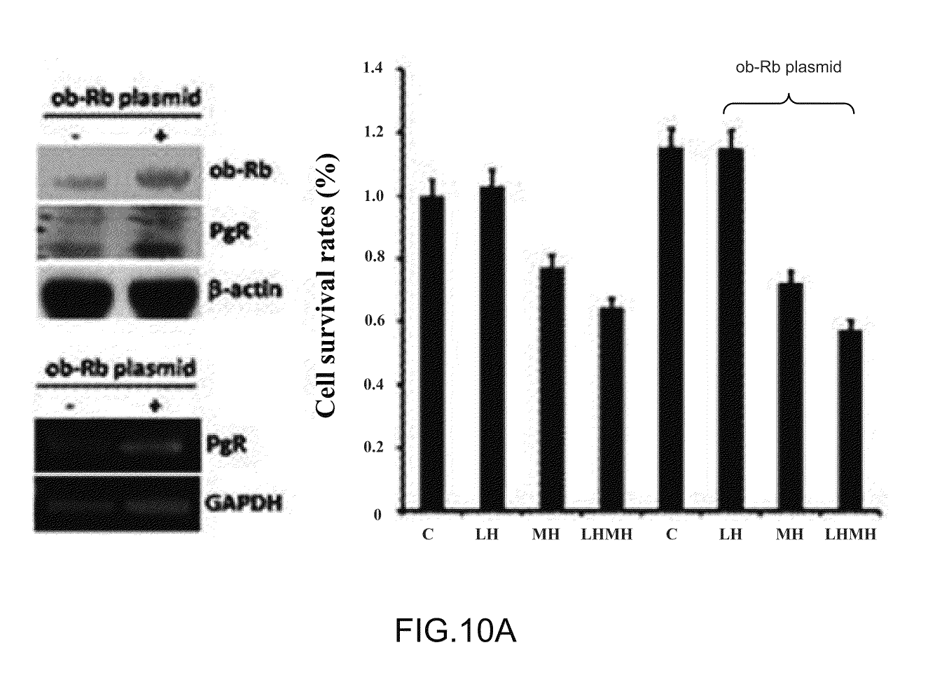

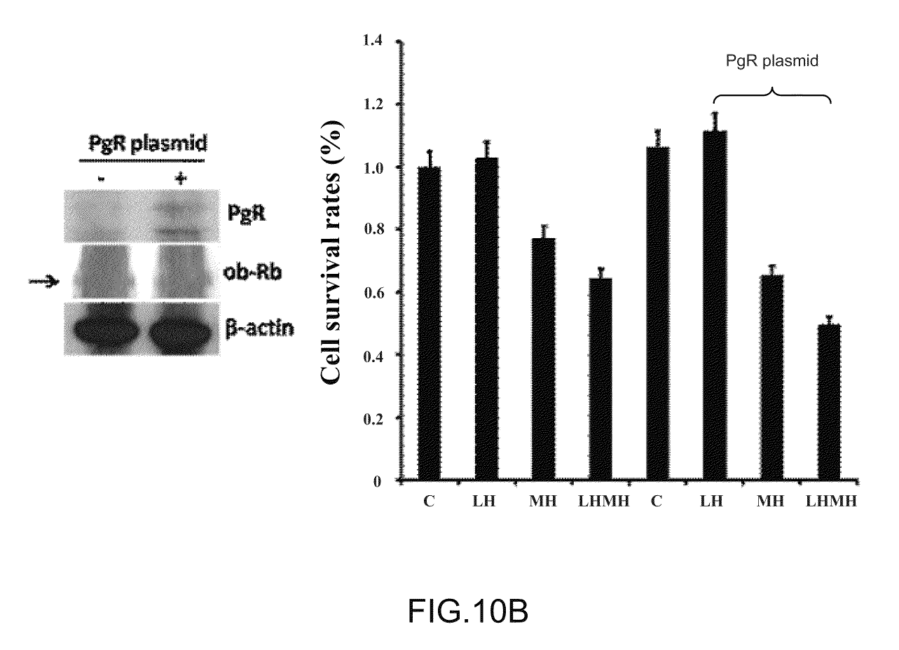

Please refer to FIG. 10A and FIG. 10B, which are views showing apoptosis of cells on increasing expression of leptin receptor and expression of progesterone receptor respectively. As shown in the figures, the preferred embodiment makes ob-Rb plasmid transfected to cells to be cultured a day and is cultured in a serum-free medium for the next day. Then, the cells are processed with MPA+leptin for 24 hours to be analyzed through XTT assay. As results show in FIG. 10A, when cells have very much expression of long-form Ob-R, MPA's treatment effect is obviously enhanced. Besides, the long-form Ob-R enhances expression of the progesterone receptor. Again, the preferred embodiment makes PgR plasmid transfected to cells to be cultured a day and is cultured in a serum-free medium for the next day. Then, the cells are processed with MPA+leptin for 24 hours to be analyzed through XTT assay. As results show in FIG. 10B, treatment effect of MPA is obviously enhanced as well when many progesterone receptors are expressed. Thus, it is confirmed that MPA+leptin works on leptin receptor directly with expression of the progesterone receptor indirectly improved, where MPA's treatment effect is thus further enhanced.

### [State 10] Enhanced Suppression on JAK/STAT Signal Path by MPA+Leptin

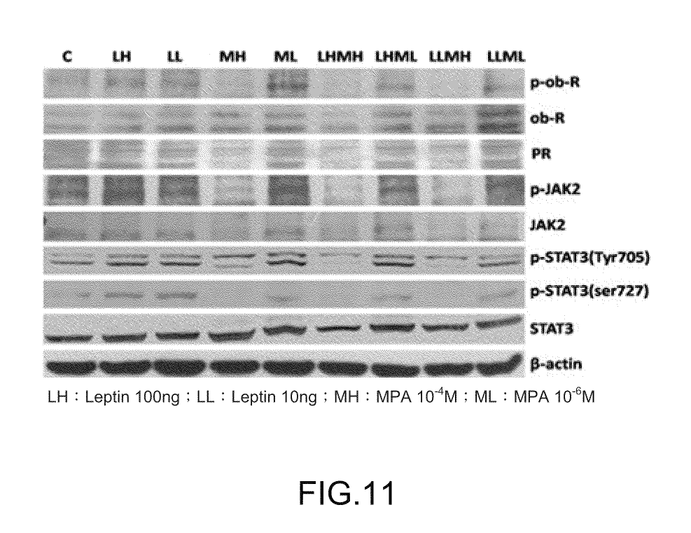

Please refer to FIG. 11, which is a view showing expression of JAK/STAT path after applied with a drug. As shown in the figure, the preferred embodiment analyzes related proteins of liver cancer cells through western blotting after applied with MPA+leptin for 24 hours. As results show, p-ob-R, ob-R and PgR are all suppressed; and furthermore, the suppression is better than that obtained after applied with MPA only. In addition, proteins related to the JAK2/STAT3 path are suppressed too. Hence, it is confirmed that MPA suppresses progesterone receptor and related protein; and, MPA+leptin has a better suppression on them.

### [State 11] Enhanced Suppression on MAPK Signal Path by MPA+Leptin

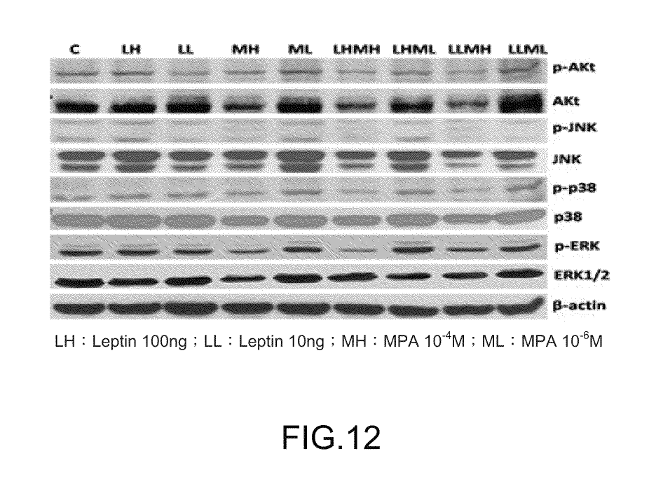

Please refer to FIG. 12, which is a view showing expression of MAPK path after applied with a drug. As shown in the figure, after proteins for the JAK2/STAT3 path are suppressed, the preferred embodiment analyzes expression of the proteins through western blotting. As results show, p-JNK, JNK, p-ERK1/2, ERK1/2, p-p38 and p38 are all suppressed. Hence, it is confirmed that MPA not only suppresses related proteins of leptin receptor but also those of the signal paths for suppressing the growth of liver cancer cells, while leptin further enhances MPA's suppression.

[State 12] Expression of Proteins of JAK/STAT Path After Applied with the Drug for a Short Time

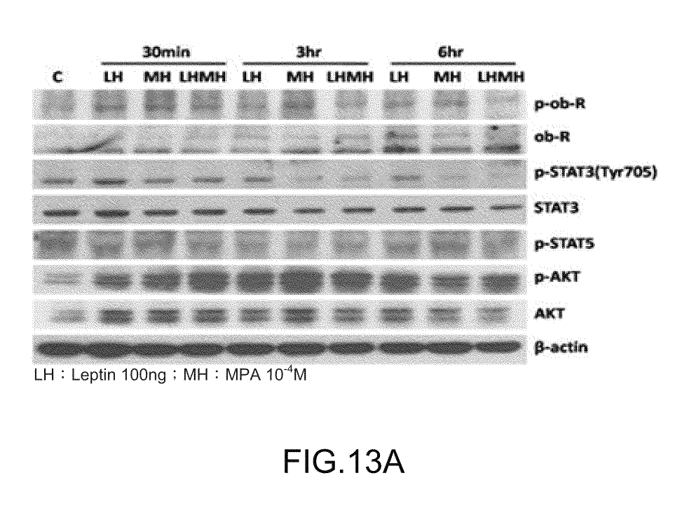

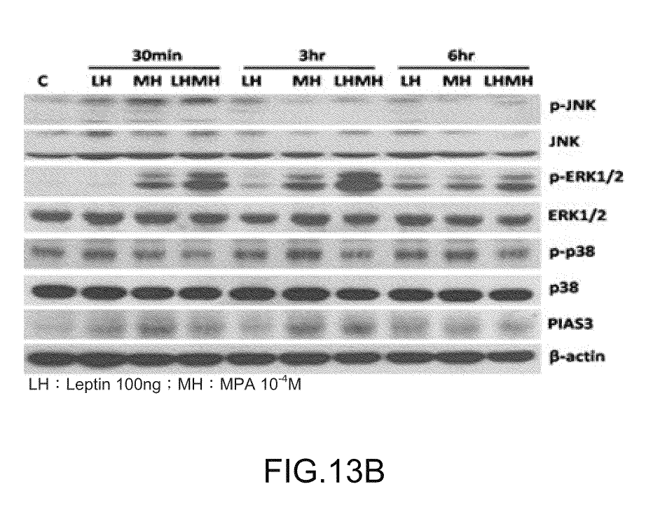

Please refer to FIG. 13A and FIG. 13A, which are views showing expressions of proteins of JAK/STAT path and proteins of MAPK path after applied with a drug for a short time. As shown in the figures, the preferred embodiment processes liver cancer cells with high-dose leptin, high-dose MPA and high-dose leptin combined with high-dose MPA for 30 minutes. Then, related JAK/STAT proteins are collected for analyzing their expressions through western blotting. As results show, p-ob-R is decreased by MPA+leptin at 30 minutes and the degradation is more obvious as time passes by. Besides, as shown in FIG. 13A, p-STAT3 (Tyr705) is started to be decreased at 3 hours, where the degradation by using MPA+leptin is better than that obtained by using MPA or leptin only. Concerning related proteins for MAPK path, p-JNK is obviously suppressed at 3 hours; and, p-ERK1/2 and PIAS3 are increased at 30 minutes and 3 hours by using MPA+leptin, as shown in FIG. 13B.

[State 13] Suppressed p-STAT3 by p-ERK1/2 Activation and PIAS3 Expression

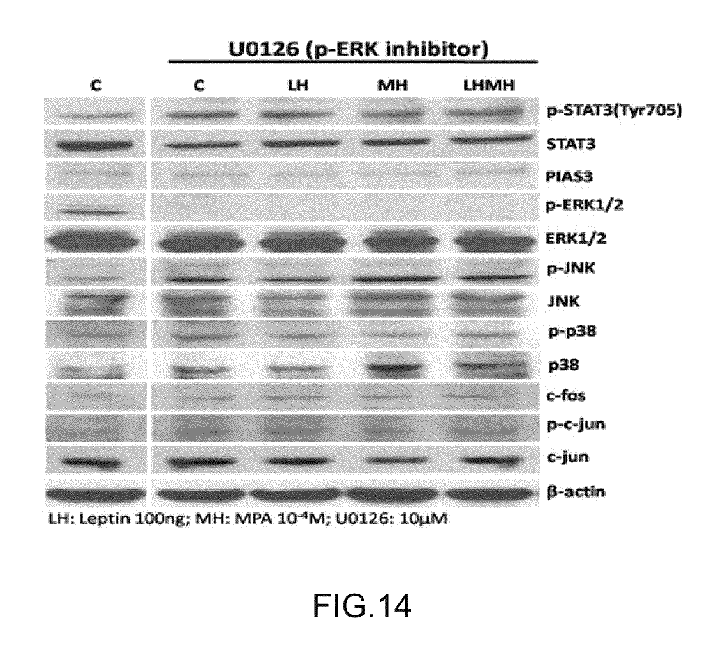

Please refer to FIG. 14, which is a view showing reduced p-STAT3 degradation by suppressing the ERK1/2 activation and the PIAS3 expression. As shown in the figure, a relationship between activated ERK1/2 and suppressed p-STAT3 shows that activated ERK1/2 suppresses p-STAT3. That is, PIAS3 is activated to suppress p-STAT3 for cell apoptosis (as shown in FIG. 13B). The preferred embodiment uses a 10⁻⁶M p-ERK1/2 inhibitor (U0126) for 4 hours before a drug is applied; then, liver cancer cells are applied with the drug of MPA+leptin for 3 hours to collect cellular proteins for analyzing expression of related proteins through western blotting. As results show, phosphorylation of ERK1/2 is suppressed; and PIAS3 is not induced or thus activated, where expression of p-STAT3 (Tyr705) is suppressed. As a conclusion, MPA activates ERK1/2 to indirectly induce PIAS3 expression for suppressing p-STAT3 (Tyr705) for cell apoptosis, where leptin enhances activation of ERK1/2 with MPA for increasing cell apoptosis.

### [State 14] Enhanced Suppression on Growth by Overexpression of Leptin in Cells

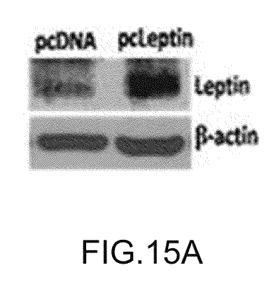

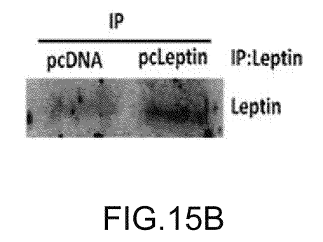

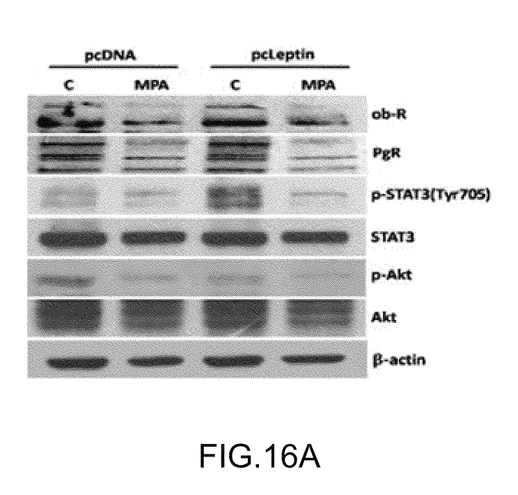

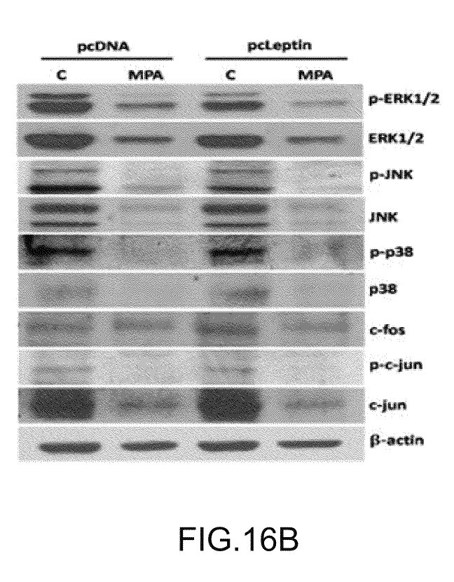

Please refer to FIG. 15A to FIG. 16B, which are views showing cells containing very much leptin within; suppressed growth of the cells by using MPA; and STAT3- and MAPK-signal paths of the cells after MPA is applied. As shown in the figures, while leptin is added from outside in state 13 to suppress liver cancer cells with MPA+leptin, the preferred embodiment here transfers expression plasmid of leptin (pcLeptin) to liver cancer cells to be cultured for a day; and a serum-free medium is used for the next day. Then, after the cells are processed with 10⁻⁴M MPA for 24 hours, cell growth is analyzed through XTT assay. In FIG. 15A, leptin gene is transferred to cells for a short time for obtaining very much leptin within the cells by themselves. After applying MPA to the cells for 24 hours, it is confirmed that MPA's suppression on liver cancer cells is enhanced by a lot of leptin self-generated within the cells. Moreover, after obtaining a lot of leptin, medium is collected for immunoprecipitation and further western blotting. In FIG. 15B, after leptin genes are transferred, cells generate leptin within by themselves. In addition, cellular proteins are collected for analyzing protein expression of STAT- and MAPK-related signal paths through western blotting. In FIG. 16A, expression plasmid of leptin (pcLeptin) is transferred to liver cancer cells to be cultured for a day; and a serum-free medium is used for the next day. Then, after the cells are processed with 10⁻⁴ M MPA for 24 hours, JAK/STAT-related proteins are analyzed through western blotting. As results show, after MPA is applied for 24 hours to the cells having a lot of leptin generated, activation of STAT3 is more obviously suppressed than that for the cells not having very much leptin generated. In FIG. 16B, related proteins for MAPK signal path are analyzed through western blotting; then, the related proteins for MAPK signal path, like ERK, JNK, p38, c-fos and c-jun, show similar results.

### [State 15] Effect of MPA+Leptin on Epithelial Cell Line of Normal Liver

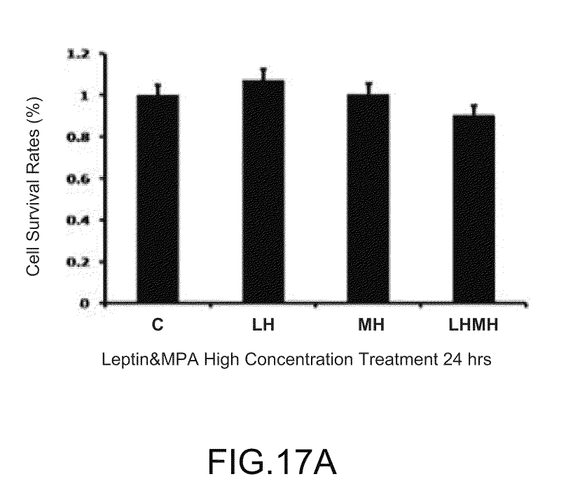

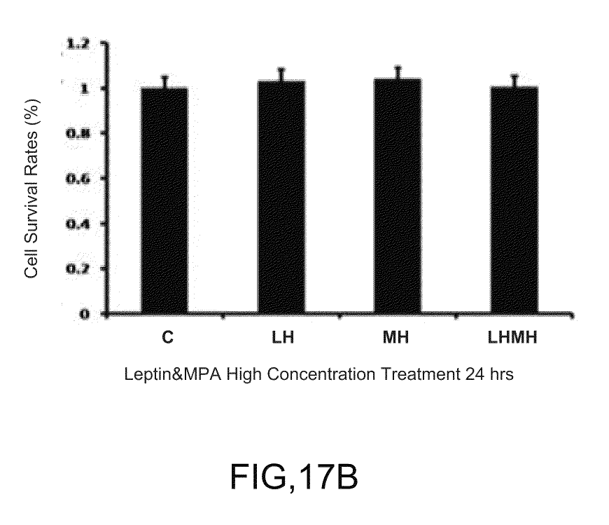

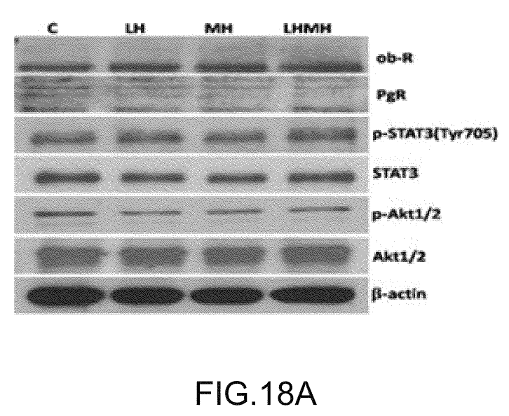

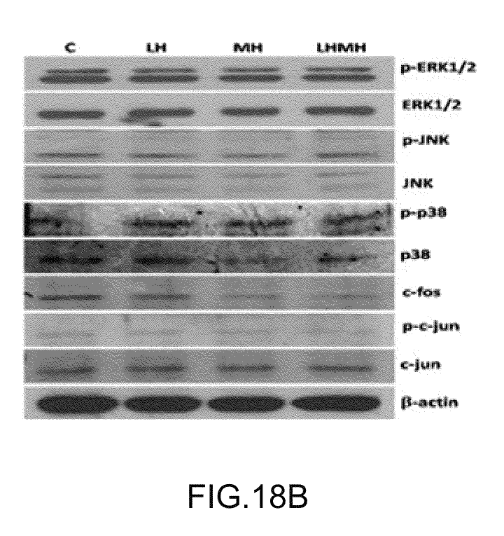

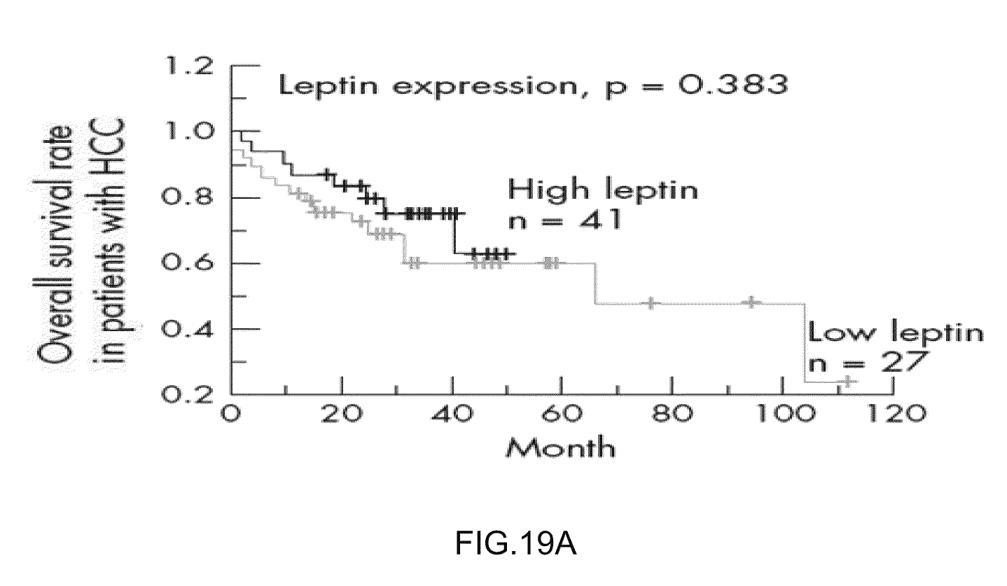

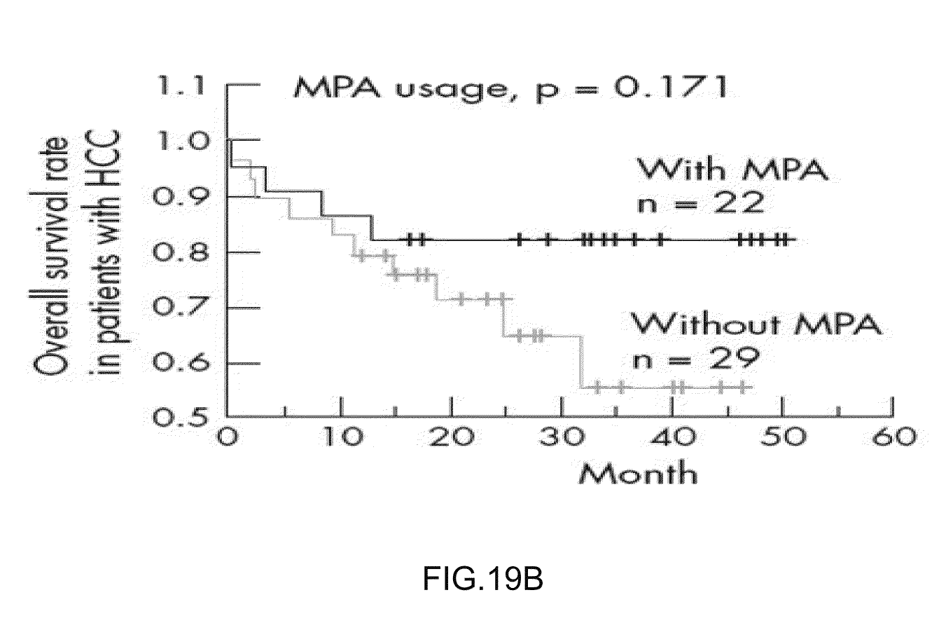

Please refer to FIG. 17A to FIG. 18B, which are views showing epithelial cell line of normal liver treated with MPA+leptin for 24 hours and 48 hours respectively; and views showing STAT3- and MAPK-related signal paths after treating cell line of normal liver with MPA+leptin. As shown in the figures, after epithelial cell line of normal liver (THLE-3) is applied with MPA+leptin for hours and 48 hours, MPA+leptin's effect on cell growth (i.e. survival rate) is analyzed through XTT assay. As results show in FIG. 17A, there is no obvious effect neither on the survival rate of the epithelial cell line of normal liver nor on poisoning the cells by using leptin only, MPA only or MPA+leptin. Besides, in FIG. 18A and FIG. 18B, protein expressions for STAT- and MAPK-related signal paths are analyzed. As results show, leptin and MPA have no obvious effect on the related signal proteins.

As shown in the above states, after liver cancer cells are applied with MPA+leptin, MPA's suppression on liver cancer cells is enhanced and effect of the suppression is better than that obtained by using MPA only. The present invention has a therapeutic drug comprising MPA combined with leptin to enhance pharmacologic effect of MPA. Or, the present invention can use 5-hydroxy-trypton (5-HTP) to heighten expression of leptin in human body before MPA is applied. Hence, the present invention helps prolong survival time of a liver cancer patient. On the other hand, when normal liver cells are applied with the present invention, they are not affected, which shows that the present invention has no bad side-effect to patients.

To sum up, the present invention is a system for curing hepatic cancer with leptin, comprising a therapeutic drug, a therapeutic procedure and a screening platform, where the therapeutic drug is a cytotoxic agent of MPA or MPA+leptin; an effective dose of the therapeutic drug is applied to patient to contact with liver cancer cells for poisoning the cells through interaction between a leptin receptor and a progesterone receptor; and the present invention has a specific pharmacologic effect on epithelial cell line of normal liver and liver cancer cells while survival rate of the patient is improved with normal liver cells not affected.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A system for curing hepatic cancer with leptin, comprising
a therapeutic drug, said therapeutic drug being selected from a group consisting of a cytotoxic agent of medroxyprogesterone acetate (MPA) and said cytotoxic agent of MPA combined with leptin (MPA+leptin),
wherein leptin is obtained by a method selected from a group consisting of adding leptin from outside of a patient and increasing physiological concentration of leptin inside said patient;
a therapeutic procedure, said therapeutic procedure applying an effective dose of said therapeutic drug to said patient to contact with liver cancer cells to enhance and accelerate poisoning liver cancer cells by MPA with leptin through an interaction of a leptin receptor and a progesterone receptor,
wherein MPA+leptin has a pharmacologic specificity on epithelial cell line of normal liver and cell line of liver cancer; and
a screening platform, said screening platform using expression of leptin as a predictive factor and a prognostic factor of treatment effect on curing said patient with MPA.

2. The system according to claim 1,
wherein said therapeutic drug reduces size and quantity of cell line of liver cancer.

3. The system according to claim 1,
wherein said system provides statistics of hepatocellular carcinoma cell line and liver cancer patients.

4. The system according to claim 1,
wherein said therapeutic drug of MPA only is directly applied to said patient to enhance a pharmacologic effect of MPA; and
wherein said patient has a high expression of leptin.

5. The system according to claim 1,
wherein said therapeutic drug of MPA+leptin is applied to said patient to enhance a pharmacologic effect of MPA; and
wherein said patient has a high expression of leptin.

6. The system according to claim 1,
wherein said therapeutic drug of MPA is applied to said patient to enhance a pharmacologic effect of MPA after increasing expression of leptin by 5-hydroxy-trypton (5-HTP); and
wherein said patient has a low expression of leptin.

7. The system according to claim 1,
wherein a higher expression of leptin obtains a better suppression by using MPA on liver cancer cells.
